(19) United States
(12) Patent Application Publication
(10) Pub. No.: US 2023/0277087 A1
(43) Pub. Date: Sep. 7, 2023

(54) RIGIDITY-ADJUSTABLE PLANTAR PRESSURE MEASURING DEVICE

(71) Applicant: Second Medical Center of Chinese PLA General Hospital, Beijing (CN)

(72) Inventors: Tianyu JIANG, Beijing (CN); Yumei XUE, Beijing (CN); Baoduo GENG, Beijing (CN); Lei HU, Beijing (CN)

(21) Appl. No.: 18/069,476

(22) Filed: Dec. 21, 2022

(30) Foreign Application Priority Data
Mar. 4, 2022 (CN) 202210212876.7

(51) Int. Cl. A61B 5/103; A61B 5/00

(52) U.S. Cl. CPC A61B 5/1038; A61B 5/6807; A61B 2562/0247

(57) ABSTRACT

A rigidity-adjustable plantar pressure measuring device can include a shoe cover body and a data processing communication unit. The shoe cover body can include a toe pressure detection part, a sole pressure detection part and a heel pressure detection part connected in sequence. The toe pressure detection part is provided with a plurality of first pressure detection components; the sole pressure detection part is provided with a plurality of second pressure detection components; and a plurality of third pressure detection components are installed on the heel pressure detection part. The data processing communication unit is installed on the shoe cover body. In the present disclosure, the device can be sleeved on the shoe body and be adjusted adaptively by the retractable component, so that the device can be adapted to shoes of all sizes. The device has good universality and ensures accuracy of the pressure detection.

### CROSS-REFERENCE TO RELATED APPLICATIONS

This application claims priority to Chinese Patent Application No. 202210212876.7, filed on Mar. 4, 2022, which is hereby incorporated by reference in its entirety.

### TECHNICAL FIELD

The present disclosure relates to the technical field of human body data information collection, in particular to a rigidity-adjustable plantar pressure measuring device.

### BACKGROUND

In modern medical rehabilitation auxiliary treatment, human gait information is one of the important bases for doctors to determine patients' rehabilitation. Detecting plantar pressure is an important part of gait analysis, and is the basis for analyzing abnormal plantar stress distribution and gait.

The traditional plantar pressure measurement is performed mainly by a pressure test plate or a plantar pressure test shoe (pad). The test system of the pressure test plate is limited to special shoes or measuring bare feet, which is not wearable and has greater limitations. Compared with the pressure test plate, the pressure test shoe (pad) is more flexible, and is not limited by the range of motion and location, but it is prone to slip largely during the detection process, which affects the detection accuracy; and the pressure test shoe cannot be adapted according to different sizes of the soles, which also has certain limitations

### SUMMARY

The present disclosure aims to provide a rigidity-adjustable plantar pressure measuring device to solve the problems that the existing plantar pressure measuring device is largely limited in application and is poor in detection accuracy.

In order to solve the above problems, the present disclosure provides a rigidity-adjustable plantar pressure measuring device sleeved on a shoe body. The device includes a shoe cover body and a data processing communication unit, where the shoe cover body includes a toe pressure detection part, a sole pressure detection part and a heel pressure detection part. A front end of the toe pressure detection part is provided with a first limit structure for containing and limiting a front end of the shoe body, a rear end of the toe pressure detection part is connected with a front end of the sole pressure detection part, and a plurality of first pressure detection components are installed on the toe pressure detection part. A rear end of the sole pressure detection part is connected with a front end of the heel pressure detection part through a retractable component, and a plurality of second pressure detection components are installed on the sole pressure detection part. A rear end of the heel pressure detection part is provided with a second limit structure for abutting against a rear end of the shoe body, and a plurality of third pressure detection components are installed on the heel pressure detection part. The data processing communication unit is installed on the shoe cover body; the data processing communication unit is connected with the first pressure detection components, the second pressure detection components, the third pressure detection components and an external data receiving terminal.

By adopting the above technical solution, the measuring device is designed as a shoe cover structure, and can be sleeved on the shoe body; the measuring device can be adjusted adaptively by the retractable component, so that the measuring device can be suitable for all sizes of shoe bodies, and then the first limit structure and the second limit structure can be used to limit and fix the shoe body so as to prevent slippage from affecting the pressure detection. The measuring device has good universality, and the accuracy of pressure detection can be ensured.

Further, the first limit structure includes a first limit cover facing an opening at a side of the sole pressure detection part, and a top of an inner wall of the first limit cover is provided with a first limit surface for limiting the shoe body.

With the above technical solution, the limit cover is configured to cover and limit the front end of the shoe body; the first limit surface is configured to prevent the shoe body from moving up relative to the measuring device, which ensures stability of the connection between the measuring device and the shoe body, and ensures the accuracy of pressure detection.

Further, the inner wall of the first limit cover is provided with a fourth pressure detection component connected with the data processing communication unit.

With the above technical solution, the fourth pressure detection component is added to detect and feedback a toe pressure, so as to provide basic data for gait analysis, thereby improving the auxiliary role of rehabilitation treatment.

Further, the retractable component is an elastic element, the sole pressure detection part is provided with a first mounting groove, the heel pressure detection part is provided with a second mounting groove, a front end of the elastic element is at least partially installed in the first mounting groove and a rear end thereof is at least partially installed in the second mounting groove.

With the above technical solution, the elastic element is used as the retractable component, which is simple in structure and easy to realize. The elastic element is arranged partially in the first mounting groove and the second mounting groove, which ensures installation stability of the elastic element and can improve connection stability of the toe pressure detection part and the sole pressure detection part.

Further, the toe pressure detection part is connected with the sole pressure detection part through a connecting mechanism which is configured to enable an angle formed between the toe pressure detection part and the sole pressure detection part to be adjustable.

With the above technical solutions, for shoes of different shapes, the connecting mechanism can be used to adjust the angle formed between the toe pressure detection part and the sole pressure detection part, so as to further improve an adaptability of the measuring device and ensure the accuracy of the pressure detection.

Further, the connecting mechanism includes a connecting bolt and a locking nut; the toe pressure detection part is provided with two first connecting lugs spaced apart, each first connecting lug is provided with a first mounting through hole, the sole pressure detection part is provided with a second connecting lug located between the two first connecting lugs, and the second connecting lug is provided with a second mounting through hole. An end of the connecting bolt passes through the two first mounting through holes and the second mounting through hole and is locked by the locking nut.

With the above technical solution, the toe pressure detection part and the sole pressure detection part are connected by matching of the connecting bolt and the locking nut, which is not simple in structure and convenient for disassembly and assembly; when the angle formed between the toe pressure detection part and the sole pressure detection part needs to be adjusted, the angle can be adjusted by directly removing the connecting bolt and the locking nut, which is convenient for operation.

Further, at least one of the first pressure detection components, the second pressure detection components and the third pressure detection components are connected with the data processing communication unit through a connection line. The toe pressure detection part is provided with a first wiring duct for the connection line to pass through, the sole pressure detection part is provided with a second wiring duct for the connection line to pass through, the heel pressure detection part is provided with a third wiring duct for the connection line to pass through, and an outlet end of the first wiring duct is arranged opposite to an inlet end of the second wiring duct, and an outlet end of the second wiring duct is arranged opposite to an inlet end of the third wiring duct.

With the above technical solution, the first wiring duct, the second wiring duct and the third wiring duct form a whole wiring structure, which together contain a connecting line, thereby simplifying the wiring structure.

Further, widths of the first wiring duct, the second wiring duct and the third wiring duct are greater than or equal to twice a radial dimension of the connection line.

With the above technical solution, a winding wire may be reserved for the connection line to prevent the connection line from being damaged when a distance between the sole pressure detection part and the heel pressure detection part increases.

Further, the sole pressure detection part includes a metatarsal region and a mid-foot region in a direction away from the toe pressure detection part; a number of the second pressure detection components in the metatarsal region is less than that in the mid-foot region; a number of the second pressure detection components in the mid-foot region is less than or equal to a number of the first pressure detection components in the toe pressure detection part and less than or equal to a number of the third pressure detection components in the heel pressure detection part.

With the above technical solution, according to a distribution of the plantar pressure, a distribution of all detection components is designed in an optimized mode so as to ensure reasonable layout of the pressure sensors.

Further, each side of the sole pressure detection part is provided with a first limit retaining wall for limiting a side of the shoe body; and/or each side of the heel pressure detection part is provided with a second limit retaining wall for limiting the side of the shoe body.

With the above technical solution, the stability of the connection between the shoe cover body and the shoe body can be further improved by the first limit retaining wall and the second limit retaining wall

### BRIEF DESCRIPTION OF THE DRAWINGS

In order to explain the technical solutions in the embodiments of the present disclosure or the technical solutions in the prior art more clearly, the following will briefly describe the drawings in the embodiments or the description of prior art. Apparently, the accompanying drawings in the following description are only embodiments of the present disclosure. For ordinary technicians in the field, other drawings can be obtained according to the provided drawings, without paying creative efforts.

FIG. 1 is a schematic structural diagram of a rigidity-adjustable plantar pressure measuring device according to an embodiment of the present disclosure;

FIG. 2 is a top view of the rigidity-adjustable plantar pressure measuring device according to the embodiment of the present disclosure; and

FIG. 3 is a schematic structural diagram of a data processing communication unit of the rigidity-adjustable plantar pressure measuring device according to the embodiment of the present disclosure.

### REFERENCE NUMERALS

100 shoe cover body;
110 toe pressure detection part; 111 first limit structure; 112 first pressure detection component; 113 first connecting lug; 114 first wiring duct;
120 sole pressure detection part; 121 second pressure detection component; 122 first mounting groove; 123 second connecting lug; 124 second wiring duct; 125 metatarsal region; 126 mid-foot region; 127 first limit retaining wall;
130 heel pressure detection part; 131 second limit structure; 1311 avoidance hole; 132 third pressure detection component; 133 second mounting groove; 134 third wiring duct; 135 second limit retaining wall;
140 retractable component;
150 connecting mechanism; 151 connecting bolt; 152 locking nut; and
200 data processing communication unit.

### DETAILED DESCRIPTION OF THE EMBODIMENTS

In order to make the above purposes, features and advantages of the present disclosure much easier to be understood, the specific embodiments of the present disclosure are described below with reference to the accompanying drawings. It should be understood that the specific embodiments described here serve only to illustrate and explain the present disclosure rather than limiting the present disclosure.

Based on the problems that the traditional plantar pressure detection structure cannot be worn and have large limitations, the present disclosure provides a wearable and rigidity-adjustable plantar pressure measuring device that can be sleeved on a shoe body, to solve the above disadvantages of the traditional plantar pressure detection structure.

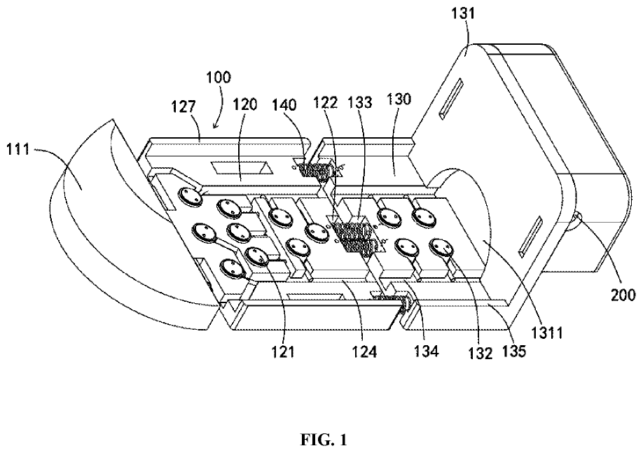

As shown in FIG. 1, the rigidity-adjustable plantar pressure measuring device provided in this embodiment mainly includes a shoe cover body 100 and a data processing communication unit 200. In this embodiment, the shoe cover body 100 is configured as a basis for pressure detection and as a mounting base for the data processing communication unit. The data processing communication unit 200 in this embodiment is mainly configured to receive a detected pressure signal, process the detected pressure signal, and transmit the processed data to an external data receiving terminal (upper computer).

In the present embodiment, the shoe cover body 100 includes a toe pressure detection part 110, a sole pressure detection part 120, and a heel pressure detection part 130. Main bodies of the toe pressure detection part 110, the sole pressure detection part 120, and the heel pressure detection part 130 can be of a plate-like structure, as shown in figures. In this embodiment, a front end of the toe pressure detection part 110 is provided with a first limit structure 111 for containing and limiting a front end of the shoe body. A rear end of the toe pressure detection component 110 is connected with a front end of the sole pressure detection part 120, and a plurality of first pressure detection components 112 are installed on the toe pressure detection part 110. In this embodiment, a rear end of the sole pressure detection part 120 and a front end of the heel pressure detection part 130 are connected through a retractable component 140, and a plurality of second pressure detection components 121 are installed on the sole pressure detection part 120. A rear end of the heel pressure detection part 130 in this embodiment is provided with a second limit structure 131 for abutting against a rear end of the shoe body, and a plurality of third pressure detection components 132 are installed on the heel pressure detection part 130. In some embodiments, the first pressure detection component 112, the second pressure detection component 121 and the third pressure detection component 132 in this embodiment are all rigid pressure sensors. The rigid pressure sensors can prevent slippage, and the rigid pressure sensors can be cylindrical, and a height of each rigid pressure sensor on each detection part is no more than 10 mm to prevent inconvenient wearing.

The data processing communication unit 200 in this embodiment is installed on the shoe cover body 100. Specifically, it can be installed on a rear side of the second limit structure 131 and protected by a shell thereof. Alternatively, other installation methods or installation locations can also be used, which are not described one by one. The data processing communication unit 200 in this embodiment is connected with the first pressure detection part 112, the second pressure detection part 121, the third pressure detection part 132 and an external data receiving terminal.

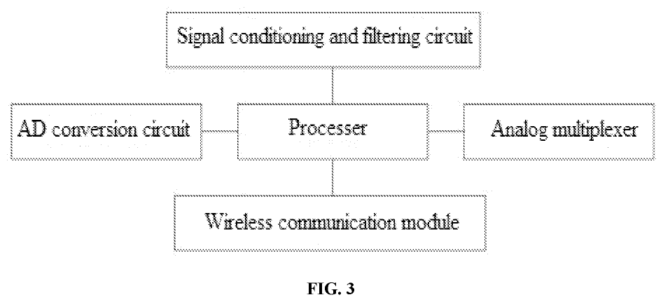

As shown in FIG. 3, the data processing communication unit 200 in this embodiment preferably includes a signal conditioning and filtering circuit, an AD conversion circuit, an analog multiplexer, a processor and a wireless communication module. The signal conditioning filtering circuit in this embodiment is configured to perform signal conditioning and filtering amplification on pressure data detected by the first pressure detection component 112, the second pressure detection component, the third pressure detection component 132, etc. The AD conversion circuit in this embodiment is configured to convert a filtered and amplified analog signal into a digital signal, and the analog multiplexer in this embodiment is configured to conduct sampling channels sequentially. Finally, the processor in this embodiment is configured to digitize, save and package the data output by the signal conditioning and filtering circuit, and send the processed data to the upper computer through the wireless communication module which may be Bluetooth.

During detection, the rigidity-adjustable plantar pressure measuring device is sleeved on the shoe body. When an overall length of the device needs to be adjusted to adapt to different sizes of shoe bodies, the device is correspondingly adjusted through the retractable component 140. After the device is fixed with the shoe body, the shoe body is limited and fixed by the first limit structure 111 and the second limit structure 131 to prevent slippage which affects the pressure detection. Then, a subject may wear the plantar pressure measuring device and perform complete gait movement. The first pressure detection component 112, the second pressure detection component 121 and the third pressure detection component 132 are used to perform static and dynamic pressure detection, and the detected data is processed and converted by the data processing communication unit, and finally sent to the external data receiving terminal, i.e. the upper computer. The whole detection process is convenient, fast and stable, which not only has good universality, but also can ensure accuracy of pressure detection.

In combination with FIG. 1, the first limit structure 111 of the embodiment is configured as a first limit cover which faces an opening at one side of the sole pressure detection part 120. A top of an inner wall of the first limit cover is provided with a first limit surface for limiting the shoe body (not shown in the figures). Preferably, the first limit surface can be parallel to the toe pressure detection component 110 or at a certain angle relative to the toe pressure detection component 110 to adapt to a structure of the shoe body. After the rigidity-adjustable plantar pressure measuring device is mounted to the shoe body in this embodiment, the front end of the shoe body is covered and limited with the first limit cover, and the first limit surface is configured to prevent the shoe body from moving up relative to the plantar pressure measuring device, thereby ensuring stability of the connection between the plantar pressure measuring device and the shoe body, and ensuring the accuracy of the pressure detection.

Further, a fourth pressure detection component (not shown in the figure) can also be installed on the inner wall of the first limit cover in this embodiment. The fourth pressure detection component is connected to the data processing communication unit 200. The added fourth pressure detection component has the same structure as the pressure detection components above, it can detect and feedback side pressure at the toe, and further provide basic data for gait analysis, thereby improving the auxiliary role of rehabilitation treatment.

The second limit structure 131 in this embodiment is a plate-like structure vertically mounted on the heel pressure detection component 130. A lower part of the second limit structure 131 can be provided with an avoidance hole 1311, which is used to accommodate partial structure of the rear end of the shoe body.

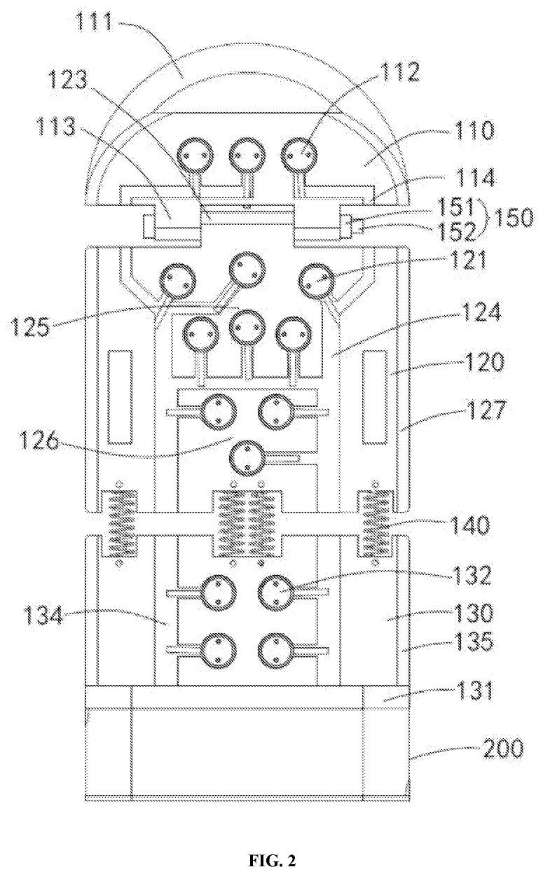

As shown in FIG. 1 and FIG. 2, the retractable component 140 of this embodiment is an elastic element such a spring or an elastic piece. Accordingly, in this embodiment, the sole pressure detection part 120 is provided with a first mounting groove 122, and the heel pressure detection part 130 is provided with a second mounting groove 133. A front end of the elastic element is at least partially installed in the first mounting groove 122, and a rear end of the elastic element is at least partially installed in the second mounting groove 133. That is, a part of the elastic element close to the sole pressure detection part 120 is located in the first mounting groove 122, and a part of the elastic element close to the heel pressure detection part 130 is located in the second mounting groove 133. Specific installation method of the elastic element is not limited, as long as it can be fixed.

In this way, the elastic element is used as the retractable component 140, which is simple in structure and easy to realize. The elastic element is located partially in the first mounting groove 122 and the second mounting groove 133, which ensures installation stability of the elastic element and can improve connection stability of the toe pressure detecting part 110 and the sole pressure detecting part 120.

As shown in FIG. 1 and FIG. 2, the toe pressure detection part 110 and the sole pressure detection part 120 are connected through a connecting mechanism 150, and the connecting mechanism 150 in this embodiment is configured to enable an angle formed between the toe pressure detection component 110 and the sole pressure detection component 120 to be adjustable. In this way, for shoes of different shapes, an angle formed between the toe pressure detection part 110 and the sole pressure detection part 120 can be adjusted so that an adaptability of the device to shoes of different shapes is further improved, ensuring the accuracy of pressure detection.

In combination with FIG. 2, the connecting mechanism 150 in this embodiment preferably includes a connecting bolt 151 and a locking nut 152. In order to install the connecting bolt 151, the toe pressure detection part 110 in this embodiment is provided with two first connecting lugs 113 spaced apart, and the first connecting lug 113 is provided with a first mounting through hole (not identified in the figure). Accordingly, the sole pressure detection part 120 in this embodiment is provided with a second connecting lug 123 located between the two first connecting lugs 113, and the second connecting lug 123 is provided with a second mounting through hole (not identified in the figure). An axial direction of the first mounting through hole and the second mounting through hole is perpendicular to a length direction of the shoe cover body 100. During assembling, an end of the connecting bolt 151 in this embodiment passes through the two first mounting through holes and the second mounting through hole, and is locked by the locking nut 152, thus completing connection and fixation of the toe pressure detection part 110 and the sole pressure detection part 120. The connecting mechanism of the present disclosure is simple in structure and convenient in assembling and disassembling; furthermore, when the angle formed between the toe pressure detection part 110 and the sole pressure detection part 120 needs to be adjusted, directly removing the connection between the connecting bolt 151 and the locking nut 152 can adjust the angle formed between the toe pressure detection part 110 and the sole pressure detection part 120, and after adjustment, the connecting bolt 151 can be locked again through the locking nut 152, which is convenient for operation.

The structure of the connecting mechanism 150 in this embodiment is not limited to this, and other mechanisms that can adjust an angle are also within an optional scope of this embodiment, such as forming the connecting mechanism 150 through a hinge and a locking part which can brake the hinge or release the hinge.

In combination with FIG. 2, in order to ensure transmission stability of pressure detection data and reduce costs, in this embodiment, each pressure detection component is designed to be connected with the data processing communication unit 200 through a line connection, and the wiring structure is optimized. Specifically, at least one of the first pressure detection component 112, the second pressure detection component 121 and the third pressure detection component 132 is connected with the data processing communication unit 200 through a connection line (not shown in the figure). For example, as shown in the figure, the two first pressure detection components 112, a plurality of the second pressure detection components 121 and a plurality of the third pressure detection components 132 are jointly connected with one connection line to route on a left side, and another first pressure detection component 112, another plurality of the second pressure detection components 121 and another plurality of the third pressure detection components 132 are jointly connected with one connection line to route on a right side.

Correspondingly, in this embodiment, a first wiring duct 114 is provided at a left side of the toe pressure detection part 110 for the left connection line to pass through, a second wiring duct 124 is provided at a left side of the sole pressure detection component 120 for the left connection line to pass through, a third wiring duct 134 is provided at a left side of the heel pressure detection component 130 for the left connection line to pass through, and it is the same for the right side. Further, on the same side, an outlet end of the first wiring duct 114 is arranged opposite to an inlet end of the second wiring duct 124; and an outlet end of the second wiring duct 124 is arranged opposite to an inlet end of the third wiring duct 134, so that the first wiring duct 114, the second wiring duct 124 and the third wiring duct 134 form an integral wiring structure which jointly contains one connection line, simplifying the wiring structure.

In addition, in this embodiment, in order to facilitate a winding wire to be reserved for the connection line and prevent the connection line from being damaged when a distance between the sole pressure detection component 120 and the heel pressure detection component 130 increases, widths of the first wiring duct 114, the second wiring duct 124 and the third wiring duct 134 are greater than or equal to twice a radial size of the connection line.

In combination with FIG. 2, in this embodiment, a number of and a distribution of the pressure detection components on each pressure detection part are designed so that the number and the distribution are more consistent with distribution of human plantar pressure, improving the accuracy of detection. Specifically, according to the fact that the plantar pressure of the human body is mainly distributed in three regions in a static state: heel, forefoot and toe, and the fact that pressure values in these three regions may also change significantly during the human body's movement, the pressure distribution of the forefoot can be further divided.

Therefore, in this embodiment, in a direction away from the toe pressure detection part 110, the sole pressure detection part 120 is divided into a metatarsal region 125 and a mid-foot region 126 which are adjacent to each other; a number of the second pressure detection components 121 in the metatarsal region 125 is less than that in the mid-foot region 126. A number of the second pressure detection components 121 in the mid-foot region 126 is less than or equal the number of the first pressure detection components 112 in the toe pressure detection part 110, and less than or equal to a number of the third pressure detection components 132 in the heel pressure detection part 130. In this embodiment, three first pressure detection components 112 are provided in the toe pressure detecting part 110, and five second pressure detecting components 121 are provided in the metatarsal region 125, three pressure detection components are provided in the mid-foot region 126, and four pressure detection components are provided in the heel region.

Using the above technical solution, the distribution of all detection components is optimized according to the plantar pressure distribution, to ensure the reasonable layout of the pressure sensors and provide a good data basis for follow-up medical rehabilitation auxiliary treatment and biped robot research.

As shown in FIG. 1, in this embodiment, a first limit retaining wall 127 for limiting a side of the shoe body is also provided on each side of the sole pressure detection part 120. Similarly, a second limit retaining wall 135 for limiting the side of the shoe body is provided on each side of the heel pressure detection part 130. Therefore, the stability of the connection between the shoe cover body 100 and the shoe body can be further improved by the first limit retaining wall 127 and the second limit retaining wall 135.

Further, in this embodiment, a foot side pressure detection part (not shown in the figure) may be installed on each side of the sole pressure detecting part 120, the foot side pressure detection part is installed on the sole pressure detection part 120 through a mounting mechanism, a fifth pressure detection component is installed on a side of the foot side pressure detection part 120 facing the sole pressure detection part 120, and the fifth pressure detection component is connected with the data processing communication unit 200. The mounting mechanism is configured to adjust a relative distance between two foot side pressure detection parts, such as a retractable rod used as the mounting mechanism.

In the description of this embodiment, it should be noted that those skilled in the art can understand that all or part of the processes in the above embodiments can be completed by a control device instructed by a computer program. The program can be stored in a computer readable storage medium. When executing the program, it can include the processes in the above embodiments. The storage medium can be a memory, a disk, a CD and the like.

Although the present disclosure is disclosed as above, the present disclosure is not limited to this. Any person skilled in the art can make various changes and modifications without departing from the spirit and scope of the disclosure. Therefore, the scope of protection of the present disclosure shall be subjected to the scope defined in the claims.

Finally, it should also be noted that in the present disclosure, relational terms such as first and second are only used to distinguish one entity or operation from another entity or operation, and not used to require or imply any such actual relationship or order between these entities or operations. Moreover, the term “including”, “comprising” or any other variant thereof is intended to cover non-exclusive inclusion, so that a process, method, article or device including a series of elements not only includes those elements, but also includes other elements not explicitly listed, or also includes elements inherent to such process, method, article or device. Without further restrictions, the element defined by the statement “including a . . . ” does not exclude that there is other identical element in the process, method, article or device including the element.

In this specification, each embodiment is described in a progressive manner. Each embodiment focuses on the differences with other embodiments, and the same and similar parts of each embodiment can be referred to each other.

The above description of the disclosed embodiments enables those skilled in the art to realize or use the present disclosure. A variety of modifications to these embodiments will be apparent to those skilled in the art. The general principles defined herein can be implemented in other embodiments without departing from the spirit or scope of the present disclosure. Therefore, the present disclosure will not be limited to the embodiments shown herein, but will conform to the widest scope consistent with the principles and novel features disclosed herein.

What is claimed is:

1. A rigidity-adjustable plantar pressure measuring device sleeved on a shoe body, comprising a shoe cover body and a data processing communication unit, wherein the shoe cover body comprises a toe pressure detection part, a sole pressure detection part and a heel pressure detection part;
a front end of the toe pressure detection part is provided with a first limit structure for containing and limiting a front end of the shoe body, a rear end of the toe pressure detection part is connected with a front end of the sole pressure detection part, and a plurality of first pressure detection components are installed on the toe pressure detection part;
a rear end of the sole pressure detection part is connected with a front end of the heel pressure detection part through a retractable component, and a plurality of second pressure detection components are installed on the sole pressure detection part;
a rear end of the heel pressure detection part is provided with a second limit structure for abutting against a rear end of the shoe body, and a plurality of third pressure detection components are installed on the heel pressure detection part;
the data processing communication unit is installed on the shoe cover body the data processing communication unit is connected with the first pressure detection components, the second pressure detection components, the third pressure detection components and an external data receiving terminal.

2. The rigidity-adjustable plantar pressure measuring device according to claim 1, wherein the first limit structure comprises a first limit cover facing an opening at a side of the sole pressure detection part, and a top of an inner wall of the first limit cover is provided with a first limit surface for limiting the shoe body.

3. The rigidity-adjustable plantar pressure measuring device according to claim 2, wherein the inner wall of the first limit cover is provided with a fourth pressure detection component connected with the data processing communication unit.

4. The rigidity-adjustable plantar pressure measuring device according to claim 1, wherein the retractable component is an elastic element, the sole pressure detection part is provided with a first mounting groove, the heel pressure detection part is provided with a second mounting groove, a front end of the elastic element is at least partially installed in the first mounting groove and a rear end thereof is at least partially installed in the second mounting groove.

5. The rigidity-adjustable plantar pressure measuring device according to claim 1, wherein the toe pressure detection part is connected with the sole pressure detection part through a connecting mechanism, which is configured to enable an angle formed between the toe pressure detection part and the sole pressure detection part to be adjustable.

6. The rigidity-adjustable plantar pressure measuring device according to claim 5, wherein the connecting mechanism comprises a connecting bolt and a locking nut;
the toe pressure detection part is provided with two first connecting lugs spaced apart, each of the two first connecting lug is provided with a first mounting through hole, the sole pressure detection part is provided with a second connecting lug located between the two first connecting lugs, and the second connecting lug is provided with a second mounting through hole; and
an end of the connecting bolt passes through the two first mounting through holes and the second mounting through hole and is locked by the locking nut.

7. The rigidity-adjustable plantar pressure measuring device according to claim 1, wherein at least one of the first pressure detection components, the second pressure detection components or the third pressure detection components are connected with the data processing communication unit through a connection line; and
the toe pressure detection part is provided with a first wiring duct for the connection line to pass through, the sole pressure detection part is provided with a second wiring duct for the connection line to pass through, the heel pressure detection part is provided with a third wiring duct for the connection line to pass through, an outlet end of the first wiring duct is arranged opposite to an inlet end of the second wiring duct, and an outlet end of the second wiring duct is arranged opposite to an inlet end of the third wiring duct.

8. The rigidity-adjustable plantar pressure measuring device according to claim 7, wherein widths of the first wiring duct, the second wiring duct and the third wiring duct are greater than or equal to twice a radial dimension of the connection line.

9. The rigidity-adjustable plantar pressure measuring device according to claim 1, wherein the sole pressure detection part comprises a metatarsal region and a mid-foot region adjacent to each other in sequence along a direction away from the toe pressure detection part;
a number of the second pressure detection components in the metatarsal region is less than that in the mid-foot region; and
a number of the second pressure detection components in the mid-foot region is less than or equal to a number of the first pressure detection components in the toe pressure detection part and less than or equal to a number of the third pressure detection components in the heel pressure detection part.

10. The rigidity-adjustable plantar pressure measuring device according to claim 2, wherein the sole pressure detection part comprises a metatarsal region and a mid-foot region adjacent to each other in sequence along a direction away from the toe pressure detection part;
a number of the second pressure detection components in the metatarsal region is less than that in the mid-foot region; and
a number of the second pressure detection components in the mid-foot region is less than or equal to a number of the first pressure detection components in the toe pressure detection part and less than or equal to a number of the third pressure detection components in the heel pressure detection part.

11. The rigidity-adjustable plantar pressure measuring device according to claim 4, wherein the sole pressure detection part comprises a metatarsal region and a mid-foot region adjacent to each other in sequence along a direction away from the toe pressure detection part;
a number of the second pressure detection components in the metatarsal region is less than that in the mid-foot region; and
a number of the second pressure detection components in the mid-foot region is less than or equal to a number of the first pressure detection components in the toe pressure detection part and less than or equal to a number of the third pressure detection components in the heel pressure detection part.

12. The rigidity-adjustable plantar pressure measuring device according to claim 5, wherein the sole pressure detection part comprises a metatarsal region and a mid-foot region adjacent to each other in sequence along a direction away from the toe pressure detection part;
a number of the second pressure detection components in the metatarsal region is less than that in the mid-foot region; and
a number of the second pressure detection components in the mid-foot region is less than or equal to a number of the first pressure detection components in the toe pressure detection part and less than or equal to a number of the third pressure detection components in the heel pressure detection part.

13. The rigidity-adjustable plantar pressure measuring device according to claim 6, wherein the sole pressure detection part comprises a metatarsal region and a mid-foot region adjacent to each other in sequence along a direction away from the toe pressure detection part;
a number of the second pressure detection components in the metatarsal region is less than that in the mid-foot region; and
a number of the second pressure detection components in the mid-foot region is less than or equal to a number of the first pressure detection components in the toe pressure detection part and less than or equal to a number of the third pressure detection components in the heel pressure detection part.

14. The rigidity-adjustable plantar pressure measuring device according to claim 7, wherein the sole pressure detection part comprises a metatarsal region and a mid-foot region adjacent to each other in sequence along a direction away from the toe pressure detection part;
a number of the second pressure detection components in the metatarsal region is less than that in the mid-foot region; and
a number of the second pressure detection components in the mid-foot region is less than or equal to a number of the first pressure detection components in the toe pressure detection part and less than or equal to a number of the third pressure detection components in the heel pressure detection part.

15. The rigidity-adjustable plantar pressure measuring device according to claim 1, wherein each side of the sole pressure detection part is provided with a first limit retaining wall for limiting a side of the shoe body; and/or
each side of the heel pressure detection part is provided with a second limit retaining wall for limiting the side of the shoe body.

16. The rigidity-adjustable plantar pressure measuring device according to claim 2, wherein each side of the sole pressure detection part is provided with a first limit retaining wall for limiting a side of the shoe body; and/or
each side of the heel pressure detection part is provided with a second limit retaining wall for limiting the side of the shoe body.

17. The rigidity-adjustable plantar pressure measuring device according to claim 4, wherein each side of the sole pressure detection part is provided with a first limit retaining wall for limiting a side of the shoe body; and/or
each side of the heel pressure detection part is provided with a second limit retaining wall for limiting the side of the shoe body.

18. The rigidity-adjustable plantar pressure measuring device according to claim 5, wherein each side of the sole pressure detection part is provided with a first limit retaining wall for limiting a side of the shoe body; and/or
each side of the heel pressure detection part is provided with a second limit retaining wall for limiting the side of the shoe body.

19. The rigidity-adjustable plantar pressure measuring device according to claim 6, wherein each side of the sole pressure detection part is provided with a first limit retaining wall for limiting a side of the shoe body; and/or
each side of the heel pressure detection part is provided with a second limit retaining wall for limiting the side of the shoe body.

20. The rigidity-adjustable plantar pressure measuring device according to claim 7, wherein each side of the sole pressure detection part is provided with a first limit retaining wall for limiting a side of the shoe body; and/or
each side of the heel pressure detection part is provided with a second limit retaining wall for limiting the side of the shoe body.
